(19) United States
(12) Patent Application Publication
(10) Pub. No.: US 2023/0317222 A1
(43) Pub. Date: Oct. 5, 2023

(54) MACHINE LEARNING-BASED ELECTRONIC HEALTH RECORD PREDICTION

(71) Applicant: MatrixCare, Inc, Bloomington, MN (US)

(72) Inventors: Jessica ROCKNE, West Jordan, UT (US); Vivek KUMAR, Eden Prairie, MN (US); Doc DEVORE, Madisonville, KY (US); Barrett Gordon SELFRIDGE, Floral Park, NY (US); Adhiraj Ganpat PRAJAPATI, St. Paul, MN (US); Kedar Mangesh KADAM, Halifax, CA (US)

(21) Appl. No.: 18/175,324

(22) Filed: Feb. 27, 2023

Related U.S. Application Data
(60) Provisional application No. 63/325,964, filed on Mar. 31, 2022.

(51) Int. Cl. G16H 10/60; G06N 5/022

(52) U.S. Cl. CPC G16H 10/60; G06N 5/022

(57) ABSTRACT

Certain aspects of the present disclosure provide techniques for predicting electronic health record data using ML. This includes determining a plurality of attributes of a textual description of a patient medical examination, including: detecting the plurality of attributes based on analyzing the textual description using a first ML model trained to parse patient textual descriptions. This further includes predicting a change to an electronic health record for the patient, including: providing to a second ML model the plurality of attributes of the textual description and patient medical data for the patient, where the second ML model is trained to predict changes to patient electronic health records based on attributes of textual data relating to the patient and patient medical data. The predicted change is provided to an electronic system to change the electronic health record and affect medical treatment for the patient.

### CROSS-REFERENCE TO RELATED APPLICATIONS

This application claims priority to U.S. Provisional Patent Application No. 63/325,964, filed Mar. 31, 2022, the entire content of which is incorporated herein by reference in its entirety.

### INTRODUCTION

Aspects of the present disclosure relate to artificial intelligence and healthcare, and more specifically, to predicting electronic health record data using machine learning (ML).

As part of patient care, patient caregivers (e.g., people tasked with providing in-home and clinical healthcare to patients) frequently update a patient's electronic health record. For example, caregivers record patient symptoms, patient diagnoses, patient rehabilitation progress, and numerous other data points. Some data is entered in a structured manner, through checkboxes, drop-downs, and other structured user interface (UI) elements. But other data is entered as free text by the caregiver.

Textual data added by a caregiver to a patient's electronic health record is vitally important, but can be prone to mistakes, and is challenging to analyze and reconcile with the rest of the patient's health record. For example, caregivers with limited time may make mistakes in entering data. Caregivers may enter incorrect information, make typographical errors, or incorrectly enter data for the wrong patient. As another example, caregivers may enter data that reflects a change in the patient's health status or treatment status, but may not update corresponding separate aspects of the patient's health record necessary to implement the changes. Both of these can result in significant drawbacks, including detriments to patient care and inaccurate or inefficient electronic recordkeeping. What is needed are improved techniques to accurately, and computationally efficiently, maintain and update electronic health records.

### SUMMARY

Embodiments include a method. The method includes determining a plurality of attributes of a textual description of a patient medical examination, including detecting the plurality of attributes based on analyzing the textual description using a first machine learning (ML) model trained to parse patient textual descriptions. The method further includes predicting a change to an electronic health record for the patient, including: providing to a second ML model the plurality of attributes of the textual description and patient medical data for the patient, where the second ML model is trained to predict changes to patient electronic health records based on attributes of textual data relating to the patient and patient medical data. The predicted change is provided to an electronic system to change the electronic health record and affect medical treatment for the patient.

Embodiments further include an apparatus, including a memory, and a hardware processor communicatively coupled to the memory, the hardware processor configured to perform operations. The operations include determining a plurality of attributes of a textual description of a patient medical examination, including: detecting the plurality of attributes based on analyzing the textual description using a first ML model trained to parse patient textual descriptions. The operations further include predicting a change to an electronic health record for the patient, including: providing to a second ML model the plurality of attributes of the textual description and patient medical data for the patient, where the second ML model is trained to predict changes to patient electronic health records based on attributes of textual data relating to the patient and patient medical data. The predicted change is provided to an electronic system to change the electronic health record and affect medical treatment for the patient.

Embodiments further include a non-transitory computer-readable medium including instructions that, when executed by a processor, cause the processor to perform operations. The operations include determining a plurality of attributes of a textual description of a patient medical examination, including: detecting the plurality of attributes based on analyzing the textual description using a first ML model trained to parse patient textual descriptions. The operations further include predicting a change to an electronic health record for the patient, including: providing to a second ML model the plurality of attributes of the textual description and patient medical data for the patient, where the second ML model is trained to predict changes to patient electronic health records based on attributes of textual data relating to the patient and patient medical data. The predicted change is provided to an electronic system to change the electronic health record and affect medical treatment for the patient.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts a computing environment for predicting electronic health record data using ML, according to one embodiment.

FIG. 2 depicts a block diagram for a prediction controller for predicting electronic health record data using ML, according to one embodiment.

FIG. 3 is a flowchart illustrating predicting electronic health record data using ML, according to one embodiment.

FIG. 4 illustrates parsing patient progress notes using ML, according to one embodiment.

FIG. 5 depicts an example of a table of patient progress notes, according to one embodiment.

FIG. 6 is a flowchart illustrating training a natural language processing (NLP) ML model for parsing patient progress notes, according to one embodiment.

FIG. 7 depicts predicting electronic health record changes from parsed patient progress notes using an ML model, according to one embodiment.

FIG. 8 depicts parsed progress note data for predicting electronic health record changes using an ML model, according to one embodiment.

FIG. 9 depicts patient characteristics for use in predicting electronic health record changes from parsed patient progress notes using an ML model, according to one embodiment.

FIG. 10 depicts patient medical history for use in predicting electronic health record changes from parsed patient progress notes using an ML model, according to one embodiment.

FIG. 11 depicts historical patient progress note data for use in predicting electronic health record changes from parsed patient progress notes using an ML model, according to one embodiment.

FIG. 12 is a flowchart illustrating training an ML model for predicting electronic health record changes from parsed patient progress notes using an ML model, according to one embodiment.

FIG. 13 depicts using predicted electronic health record changes, according to one embodiment.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved prediction of electronic health record changes. As discussed above, maintaining accurate, and consistent, electronic health records is a challenging, and important, problem. Inaccurate health records can be highly detrimental to patient treatment. Further, it can be extremely difficult to maintain accurate records in electronic health recordkeeping systems.

In aspects described herein, changes to electronic health records stemming from textual data relating to the patient (e.g., patient progress notes entered by a caregiver) can be predicted automatically, using a trained ML model. For example, an ML model can be trained to use natural language processing (NLP) to parse textual patient data and identify attributes of the data. The parsed attributes can then be provided to one or more additional ML models, which can use the attributes and other patient medical data to predict changes to electronic health records. This can include predicting changes to the initial textual data (e.g., the patient progress notes) or to other patient health records (e.g., patient care plans or diagnoses). As used herein changes to electronic health records can refer to revisions to existing electronic health records, additions to electronic health records, removal of electronic health records, and any other suitable changes.

Aspects described herein provide significant advantages over conventional systems. For example, predicting changes to patient textual data (e.g., patient progress notes) and other aspects of a patient's electronic health record records (e.g., patient care plans or diagnoses), significantly improves patient treatment outcomes. Errors in progress notes, or progress notes assigned to the wrong patient, can lead to incorrect treatments for a patient. Further, changes reflected in progress notes, but not propagated to care plans or diagnoses, can lead to outdated and incorrect treatment for a patient. Predicting changes to patient textual data (e.g., patient progress notes) and other aspects of the patient's electronic health records can ensure that patients have accurate, and up to date, care at all times.

Using a trained ML model to perform these predictions also provides a significant technical advantage. For example, in an embodiment some aspects of record inconsistencies could potentially be analyzed using a specific rubric or algorithm with pre-defined rules. But this may be computationally expensive, because a very large number of rules would be needed and parsing and following the rules is computationally expensive. Further, using pre-defined rules would require this computationally expensive analysis be done at the time of the prediction, when a rapid response is likely to be needed (e.g., so that patient records can be kept up to date). Predicting changes to electronic health records automatically using a trained ML model, by contrast, is significantly less computationally expensive at the time the prediction generated. For example, the ML model can be trained during an offline training phase, when rapid response is not necessary and computational resources are readily available. The trained ML model can then be used to rapidly, and computationally cheaply, during an online inference phase to perform the prediction(s).

As another example, automatically predicting changes to electronic health records stemming from textual data relating to the patient (e.g., patient progress notes entered by a caregiver) using a trained ML model provides for a more accurate and well-defined result. In an embodiment, a care provider could manually review the textual data and the patient's electronic health record. But this leaves the risk of human error, and a lack of certainty in the accuracy of the review. Predicting changes to electronic health records using a trained ML model can both lessen the risk of human error, and provide more certainty in the level of accuracy of the prediction. Further, the prediction can itself be reviewed and refined by a care provider. This provides a starting point for the care provider with a more certain level of accuracy, and reduces the burden on the care provider to generate the prediction themselves. This is especially true because the care provider will almost certainly not have access or knowledge of all of the historical data, described further below, that can be considered at instant using one or more of the ML techniques described below.

As another example, one or more techniques described below provide an improvement to electronic health record systems. As noted above, maintaining accurate electronic health records is difficult. For example, manual review of changes to electronic health records tends to result in inaccurate records, because of the burden and difficulty of manual review. These inaccurate electronic health records can require repeated review and analysis (e.g., by caregivers or other employees of care providers). This can be computationally expensive and time-consuming. One or more techniques described herein improve this, by predicting changes to electronic health records stemming from textual data relating to the patient (e.g., patient progress notes entered by a caregiver), using a trained ML model. As described below, in an embodiment the predicted changes can be automatically provided to an electronic health record system (e.g., for a care provider or healthcare facility) and used to maintain the accuracy of the records and reduce the need for repeated review and analysis.

### Example Computing Environment

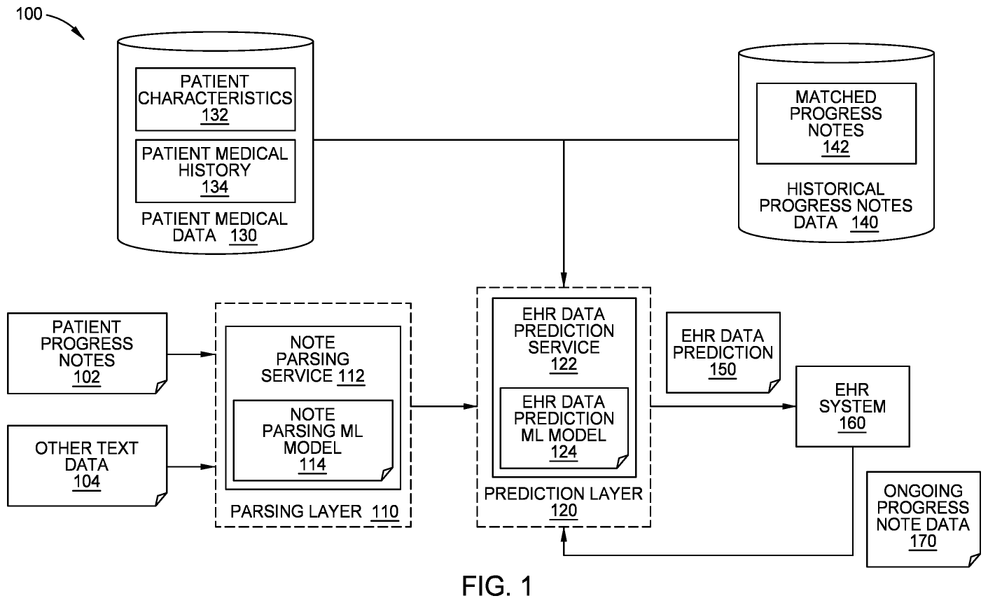

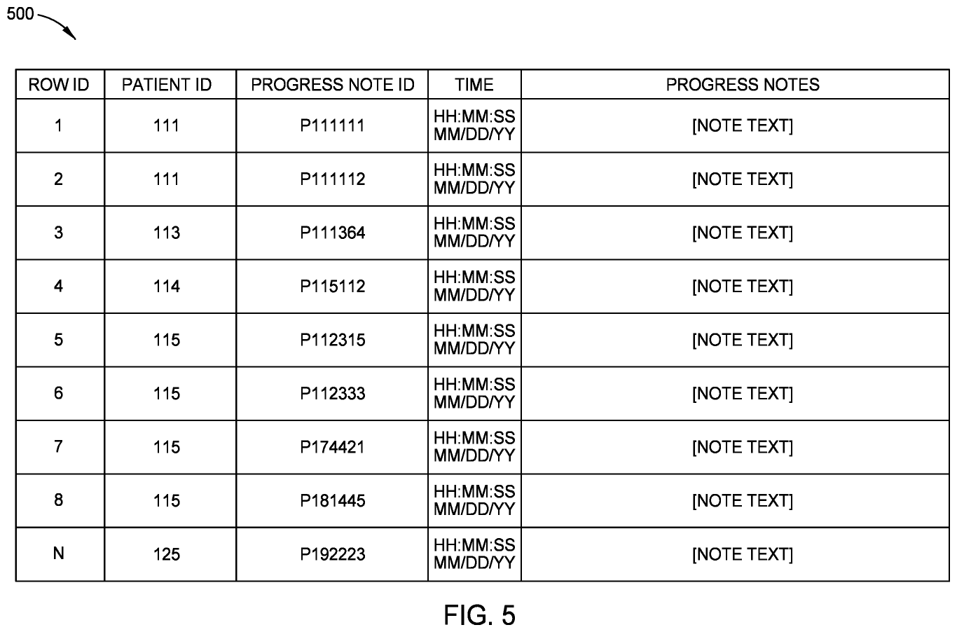

FIG. 1 depicts a computing environment 100 for predicting electronic health record data using ML, according to one embodiment. In an embodiment, one or more patient progress notes 102 are provided to a parsing layer 110. For example, the patient progress notes 102 may reflect data entered by a caregiver describing an interaction with, or examination of, a patient. The notes can be entered by a caregiver using a suitable computing device (e.g., a smartphone, tablet, laptop computer, desktop computer, wearable device, or any other suitable computing device) using a suitable input device (e.g., a keyboard, touch screen, audio microphone, or any other suitable input device). Alternatively, or in addition, the notes can be scanned from paper records (e.g., handwritten or printed notes) or imported from other sources (e.g., patient charts and records). In an embodiment, the patient progress notes stem from a medical examination of the patient and reflect the patient's current health status, treatment status and progression, prognosis, and any other suitable information. In an embodiment, the patient progress notes 102 include a portion of unstructured textual data. For example, a caregiver may enter data in a free form textual field, in addition to (or instead of) entering data through a structured user interface (e.g., checkboxes, drop-downs, and other user interface elements). This is merely an example, and the patient progress notes 102 can reflect completely unstructured textual data, partially structured textual data (e.g., data received partially from structured UI elements), or fully structured textual data (e.g., data received entirely from structured UI data). The patient progress notes 102 are discussed further, below, with regard to FIG. 5.

In an embodiment, the captured patient progress notes 102 are provided to the parsing layer 110 using a suitable communication network. For example, the patient progress notes 102 can be captured from a caregiver through a suitable user interface (e.g., a keyboard, keypad, touch sensitive interface, voice interface, or any other suitable user interface) using a suitable computing device (e.g., a smartphone, tablet, wearable device, laptop computer, desktop computer, special purpose computing device, or any other suitable comping device). The computing device can use any suitable communication network, including the Internet, a wide area network, a local area network, or a cellular network, and can use any suitable wired or wireless communication technique (e.g., WiFi or cellular communication). This is merely one example, and the captured patient progress notes 102 can be captured by a computing device and provided to the parsing layer 110 using any suitable technique (e.g., using storage medium or through a wired or wireless transmission from the camera to the computing device).

The parsing layer 110 includes a note parsing service 112, which includes a note parsing ML model 114. In an embodiment, the note parsing service 112 facilitates parsing of incoming patient data (e.g., the patient progress notes 102). As one example, the note parsing service 112 can facilitate NLP parsing of the patient progress notes 102. In this example, the note parsing ML model 114 can be a suitable NLP ML model (e.g., a deep neural network (DNN), transformer model, support vector machine (SVM), Bayesian network, maximum entropy model, conditional random field model, or any other suitable ML model).

In an embodiment, the note parsing ML model 114 can be trained to receive the patient progress notes 102, and to parse the patient progress notes 102 to identify key components of the patient progress notes. These can include patient characteristics (e.g., demographics, medications, assessments), patient medical history (e.g., past patient diagnoses and treatments), current patient diagnoses, and current patient treatments. For example, the note parsing service 112 can use the note parsing ML model 114 to categorize and classify the patient progress notes 102, tokenize the patient progress notes 102 (e.g., divide the patient progress notes text into smaller token unites), identify parts of speech in the patient progress notes 102, identify named entities in the patient progress notes 102 (e.g., patient and caregiver names, names of diagnoses, names of medications and other treatments, and other suitable named entities), identify sentiment in the patient progress notes 102, and perform any other suitable parsing of the patient progress notes. This is discussed further below with regard to FIGS. 4-6B.

In an embodiment, the patient progress notes 102 is merely one example of patient data that can be analyzed using the parsing layer 110 (e.g., using the note parsing service 112 and the note parsing ML model 114). For example, other text data 104 can be provided to the parsing layer 110. In an embodiment, the other text data 104 includes other textual data captured as part of a patient's electronic health record. For example, the other text data 104 can include text data received from other interactions with a caregiver (e.g., other free form text data received from unstructured UI), scanned copies of paper documents (e.g., paper charts or notes scanned and processed using optical character recognition (OCR)), and any other suitable text data.

In an embodiment, the note parsing service 112 can further facilitate analysis of the other text data 104. For example, the note parsing service 112 can use a note parsing ML model 114 to parse the other text data 104. Further, in an embodiment, the note parsing ML model 114 can include multiple ML models trained to parse textual data. For example, one ML model could be trained to use NLP to parse the patient progress notes 102, another ML model could be trained to parse the other text data 104 (or different ML models could be used for various other text data 104). This is merely an example, and the note parsing ML model 114 could instead be trained to use data from multiple sources (e.g., the patient progress notes 102 and other text data 104), together, to parse the text data.

In an embodiment, the parsing layer 110 provides parsed text data to a prediction layer 120. For example, the note parsing service 112 can use the note parsing ML model 114 to parse the patient progress notes 102, the other text data 104, or both. The parsing layer 110 can provide these parsed characteristics to the prediction layer 120.

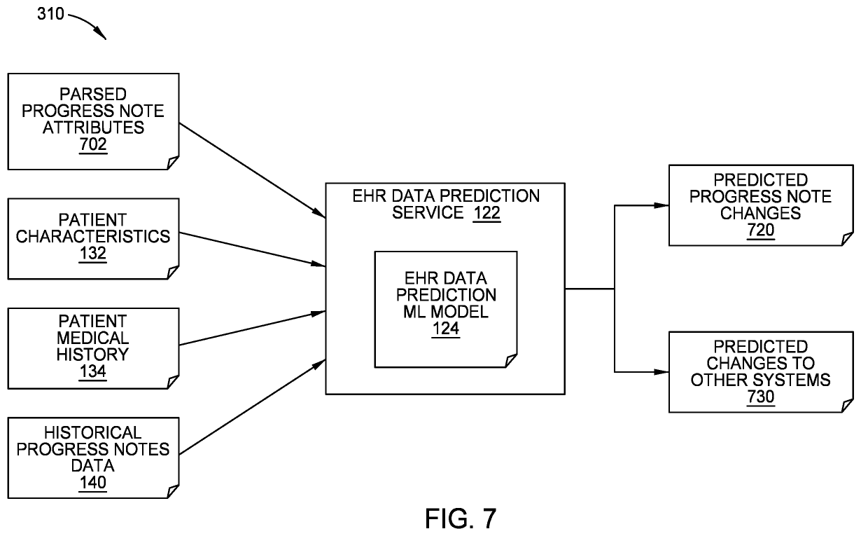

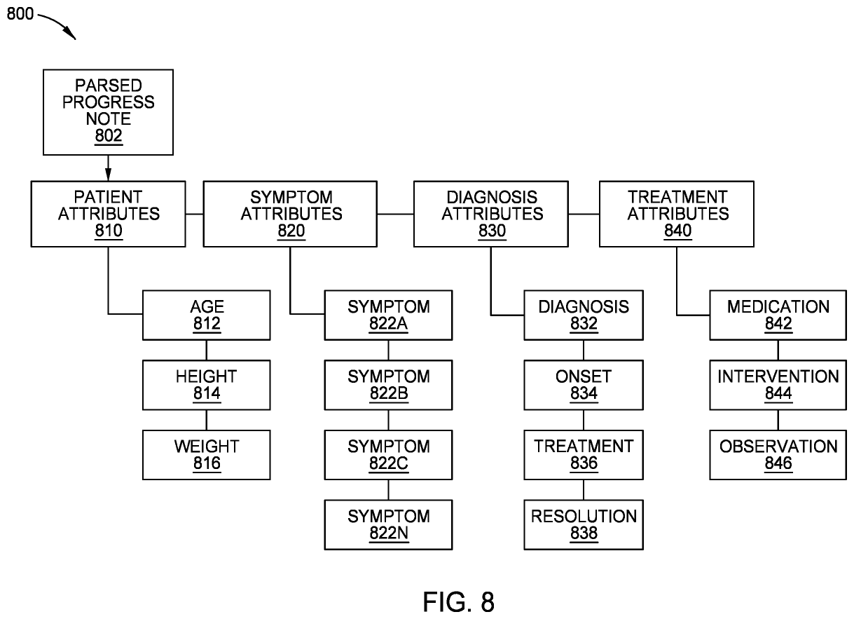

The prediction layer 120 includes an EHR data prediction service 122 and an EHR data prediction ML model 124. In an embodiment, the EHR data prediction service 122 facilitates prediction of electronic health record data for the patient (e.g., using the parsed text data received from the parsing layer 110 and patient medical data 130). For example, the EHR data prediction service 122 can use the EHR data prediction ML model 124 to determine an EHR data prediction 150. In an embodiment, the EHR data prediction 150 identifies predicted progress note changes (e.g., predicted changes to the patient progress notes 102), predicted changes to other systems (e.g., predicted changes to the patient medical data 130), or both. This is discussed further below with regard to FIG. 7.

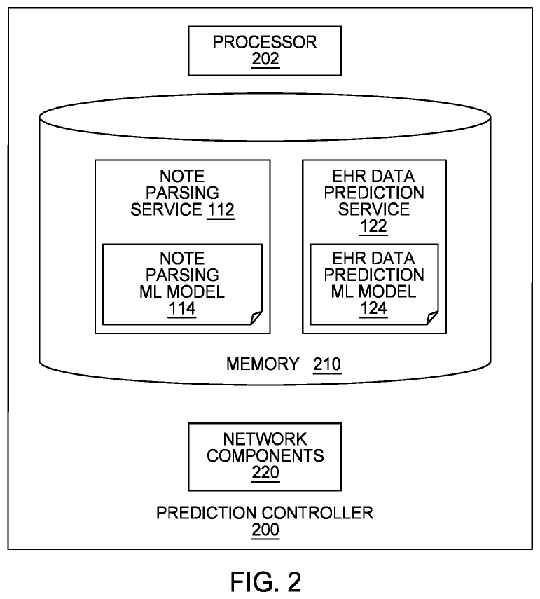

As discussed below with regard to FIG. 2, the note parsing service 112 and the prediction service 122 can be a computer software service implemented in a suitable controller (e.g., the prediction controller 200 illustrated in FIG. 2) or combination of controllers. In an embodiment each of the parsing layer 110 and prediction layer 120, and each of the note parsing service 112 and the prediction service 122, can be implemented using any suitable combination of physical compute systems, cloud compute nodes and storage locations, or any other suitable implementation. For example, the parsing layer 110 and the prediction layer 120 could be implemented using a server or cluster of servers. As another example, the parsing layer 110 and the prediction layer 120 can be implemented using a combination of compute nodes and storage locations in a suitable cloud environment. For example, one or more of the components of the parsing layer 110 and the prediction layer 120 can be implemented using a public cloud, a private cloud, a hybrid cloud, or any other suitable implementation.

As discussed above, the prediction layer 120 uses the parsed characteristics of the patient text data (e.g., the output from the parsing layer 110) to predict the patient electronic health record data. In an embodiment, however, the parsed characteristics of the patient text data detected by the parsing layer 110 are not sufficient to allow the prediction layer 120 to accurately predict the patient electronic health record data. For example, merely parsing the patient progress notes 102, themselves, may not be sufficient to identify predicted EHR data.

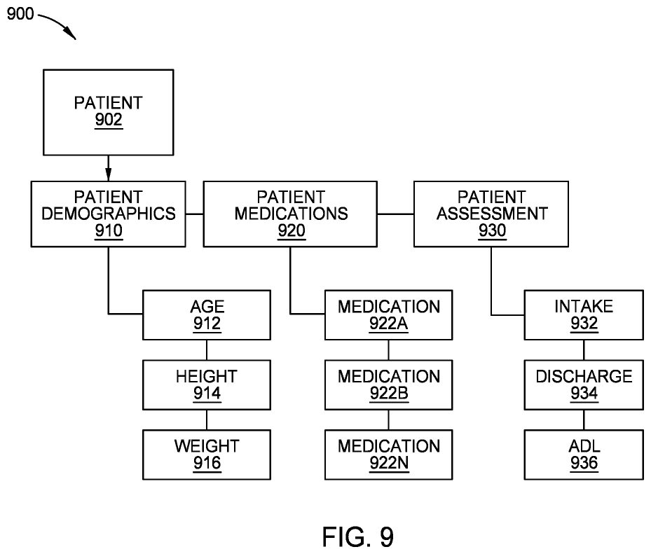

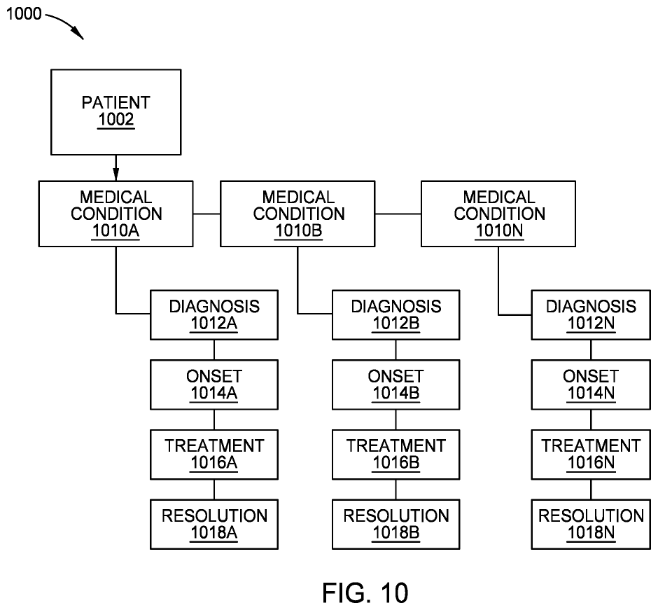

In an embodiment, the prediction layer 120 can further receive patient medical data 130 and historical progress notes data 140. For example, the patient medical data 130 can include patient characteristics 132 and patient medical history 134. In an embodiment, the patient characteristics 132 can include patient demographics (e.g., age, height, weight), patient medications (e.g., a listing of medications for the patient), patient assessment data (e.g., intake assessment data, discharge assessment data, activities of daily living (ADL) assessment data), or any other suitable patient characteristics. This is discussed further below with regard to FIG. 9. In an embodiment, the patient medical history 134 can include medical condition data (e.g., diagnosis, onset, treatment, and resolution) for any prior medical conditions. This is discussed further below with regard to FIG. 10.

In an embodiment, the historical progress notes data 140 can include data about matched progress notes 142, for various patients and various caregivers. For example, the historical progress notes data 140 can include parsed note attributes (e.g., parsed from free text progress notes or other text data) and patient medical data, matched with identified note changes and other changes. The matched progress notes 142 can include historical note changes (e.g., changes to progress notes) and historical changes to other systems (e.g., changes to other patient medical data), and any other suitable historical progress notes data. In an embodiment, the patient medical data 130 provides data about the particular patient being examined, while the historical progress notes data 140 provides data about historical progress notes and matching changes. Further, in an embodiment, the historical progress notes data 140 has had any personally identifying patient information removed.

In an embodiment, the patient medical data 130 and the historical progress notes data 140 are provided to the prediction layer 120 using a suitable communication network. For example, the patient medical data 130 and the historical progress notes 140 can be stored in one or more suitable electronic databases (e.g., a relational database, a graph database, or any other suitable database) or other electronic repositories (e.g., a cloud storage location, an on-premises network storage location, or any other suitable electronic repository). The patient medical data 130 and the historical progress notes data 140 can be provided from the respective electronic repositories to the prediction layer 120 using any suitable communication network, including the Internet, a wide area network, a local area network, or a cellular network, and can use any suitable wired or wireless communication technique (e.g., WiFi or cellular communication).

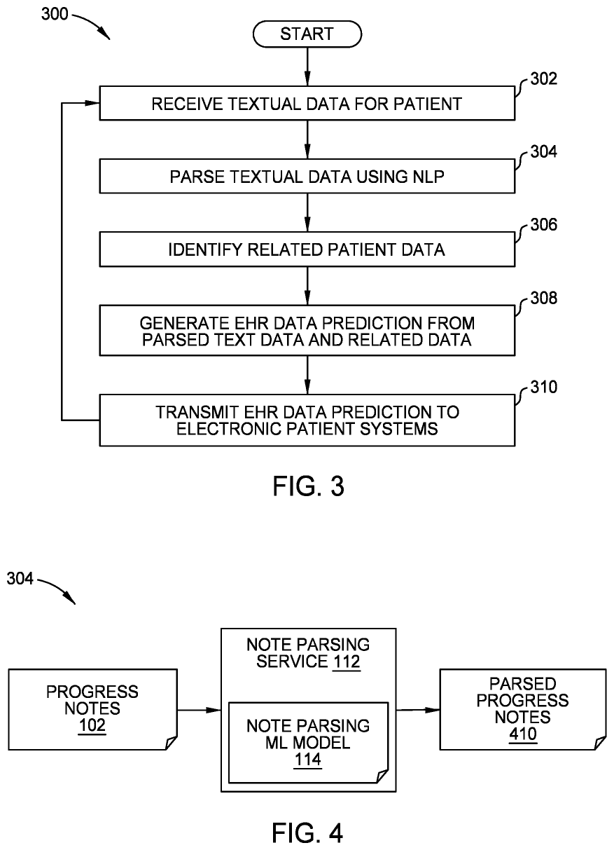

As discussed above, in an embodiment, the EHR data prediction service 122 uses the EHR data prediction ML model 124 to predict changes to progress notes, and other patient systems. For example, the EHR data prediction ML model 124 can be a suitable supervised ML model (e.g., a DNN or a transformer model) trained to generate an EHR data prediction 150 (e.g., a prediction of changes to progress notes or other patient systems) for the patient from a combination of parsed characteristics of text data (e.g., output from the parsing layer 110), patient medical data 130 and historical progress notes data 140. This is discussed further below with regard to FIG. 3. For example, the EHR data prediction ML model 124 can be selected based on initial analysis of the input data (e.g., the parsed text data characteristics, patient medical data 130, and historical progress data 140).

For example, the EHR data prediction ML model 124 can predict changes to the patient progress notes 102 (e.g., corrections to likely errors or inconsistencies in the progress notes 102). This is one example of an EHR data prediction 150. As another example, the EHR data prediction ML model 124 can predict changes to other patient systems (e.g., changes to the patient medical data 130). This is another example of an EHR data prediction 150.

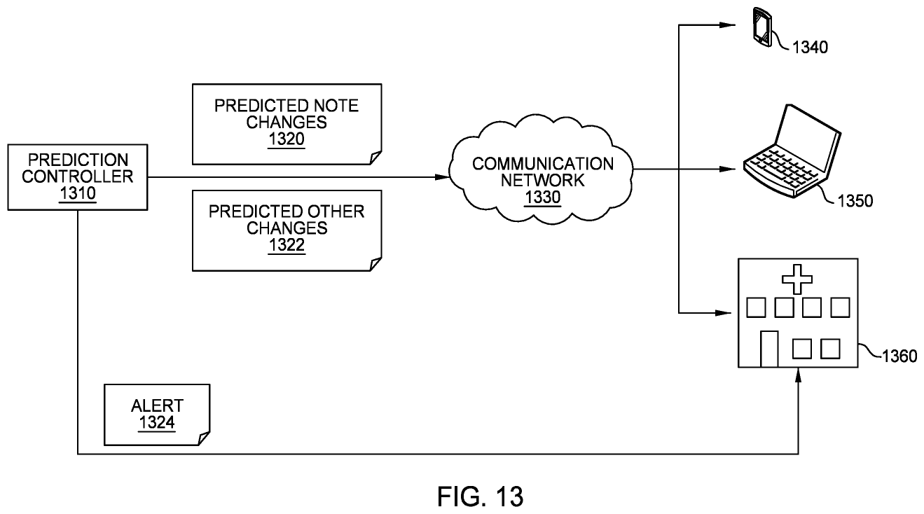

In an embodiment, the EHR data prediction 150 can be provided to an EHR system 160 (e.g., an EHR system for a medical facility or care provider. Further, in an embodiment, the EHR data prediction 150 can be provided directly to the patient's medical care provider. This is discussed further below with regard to FIG. 13. In an embodiment, the EHR data prediction 150 is provided from the prediction layer 120 to the destination (e.g., the EHR system 160) using any suitable communication network, including the Internet, a wide area network, a local area network, or a cellular network, and can use any suitable wired or wireless communication technique (e.g., WiFi or cellular communication).

In an embodiment, the EHR data prediction 150 is used to identify EHR changes that can be used by care providers to treat the patient. For example, the EHR data prediction 150 can be a prediction of changes to patient progress notes 102. These changes can improve the accuracy of the patient progress notes 102, improving the accuracy of treatment for the patient by a caregiver and improving treatment outcomes. As another example, the EHR data prediction 150 can be a prediction of changes to a patient's medical diagnosis or care plan (e.g., included in patient medical data 130). The EHR data prediction 150 can reflect a new or changed diagnosis for the patient identified from the patient progress notes 102, a new or changed treatment for the patient identified from the patient progress notes 102, or any other suitable change. This can be used to improve diagnosis and treatment for the patient, and improve patient outcomes. In either instance care providers can use the EHR data prediction 150 to treat the patient.

In an embodiment, ongoing progress note data 170 can be gathered and provided to the parsing layer 110, the prediction layer 120, or both. For example, the ongoing progress note data 170 can include additional progress notes (or other text data) for the patient, and can be gathered and maintained in suitable repository (e.g., an electronic database for the EHR system 160). This data can be used for ongoing training of the note parsing ML model or the EHR data prediction ML model (e.g., depending on the data). This data, and all training data, can be stripped of any personally identifying patient information.

In an embodiment, this ongoing progress note data 170 can be used to refine the EHR data prediction 150. For example, additional patient progress notes can be provided to the parsing layer 110 and analyzed in the same way as the patient progress notes 102 and the other text data 104 (e.g., to identify additional changes to patient medical data 130). The ongoing progress note data 170 can reflect actual changes to progress notes (e.g., made by caregivers) or additional related progress notes (e.g., stemming from a similar medical examination to the patient progress notes 102). The ongoing progress note data 170 can be assumed to be accurate, and used to refine the EHR data prediction ML model 124.

FIG. 2 depicts a block diagram for a prediction controller for predicting electronic health record data using ML, according to one embodiment. The controller 200 includes a processor 202, a memory 210, and network components 220. The memory 210 may take the form of any non-transitory computer-readable medium. The processor 202 generally retrieves and executes programming instructions stored in the memory 210. The processor 202 is representative of a single central processing unit (CPU), multiple CPUs, a single CPU having multiple processing cores, graphics processing units (GPUs) having multiple execution paths, and the like.

The network components 220 include the components necessary for the controller 200 to interface with a suitable communication network (e.g., a communication network interconnecting various components of the computing environment 100 illustrated in FIG. 1, or interconnecting the computing environment 100 with other computing systems). For example, the network components 220 can include wired, WiFi, or cellular network interface components and associated software. Although the memory 210 is shown as a single entity, the memory 210 may include one or more memory devices having blocks of memory associated with physical addresses, such as random access memory (RAM), read only memory (ROM), flash memory, or other types of volatile and/or non-volatile memory.

The memory 210 generally includes program code for performing various functions related to use of the prediction controller 200. The program code is generally described as various functional “applications” or “modules” within the memory 210, although alternate implementations may have different functions and/or combinations of functions. Within the memory 210, the note parsing service 112 facilitates parsing incoming patient text data (e.g., NLP parsing of the patient progress notes and other patient text data), using the note parsing ML model 114. This is discussed further below with regard to FIG. 4. The EHR data prediction service 122 facilitates prediction of electronic health record data for the patient (e.g., using the parsed text data created using the note parsing service 112). For example, the EHR data prediction service 122 can use the EHR data prediction ML model 124 to identify predicted progress note changes (e.g., predicted changes to patient progress notes), predicted changes to other systems (e.g., predicted changes to patient medical data), or both. This is discussed further below with regard to FIG. 7.

While the controller 200 is illustrated as a single entity, in an embodiment, the various components can be implemented using any suitable combination of physical compute systems, cloud compute nodes and storage locations, or any other suitable implementation. For example, the controller 200 could be implemented using a server or cluster of servers. As another example, the controller 200 can be implemented using a combination of compute nodes and storage locations in a suitable cloud environment. For example, one or more of the components of the controller 200 can be implemented using a public cloud, a private cloud, a hybrid cloud, or any other suitable implementation.

Although FIG. 2 depicts the note parsing service 112, the EHR data prediction service 122, the note parsing ML model 114, and the EHR data prediction ML model 124, as being mutually co-located in memory 210, that representation is also merely provided as an illustration for clarity. More generally, the controller 200 may include one or more computing platforms, such as computer servers for example, which may be co-located, or may form an interactively linked but distributed system, such as a cloud-based system, for instance. As a result, processor 202 and memory 210 may correspond to distributed processor and memory resources within the computing environment 100. Thus, it is to be understood that any, or all, of the note parsing service 112, the EHR data prediction service 122, the note parsing ML model 114, and the EHR data prediction ML model 124 may be stored remotely from one another within the distributed memory resources of the computing environment 100.

FIG. 3 is a flowchart 300 illustrating predicting electronic health record data using ML, according to one embodiment. At block 302, a note parsing service (e.g., the note parsing service 112 illustrated in FIGS. 1-2) receives textual data for a patient. For example, as discussed above in relation to FIG. 1, in an embodiment the note parsing service can receive one or more patient progress notes (e.g., the patient progress note 102 illustrated in FIG. 1), other text data (e.g., the other text data 104 illustrated in FIG. 4), or both.

At block 304, the note parsing service parses the text data using NLP (e.g., using the note parsing ML model 114 illustrated in FIGS. 1-2). For example, the note parsing service can parse patient progress notes (or other patient text data) to identify attributes of the text (e.g., items referenced in the text, the sentiment of the text, and other suitable features of the text). This can include patient attributes, diagnosis attributes, treatment attributes, and other data reflected in the text. The identified attributes are discussed further, below, with regard to FIG. 8. As discussed above in relation to the note parsing ML model 114 illustrated in FIG. 1, the parsing service can use any suitable ML model, or combination of ML models, to parse the textual data and identify features. This is discussed further below with regard to FIG. 4.

At block 306, a prediction service (e.g., the EHR data prediction service 122 illustrated in FIGS. 1-2) identifies related patient data. For example, the prediction service can identify the patient medical data 130 illustrated in FIG. 1. This can include patient characteristics (e.g., patient demographics, patient medications, patient assessment data, or any other suitable patient characteristics), patient medical history (e.g., medical condition data for any prior medical conditions), and current and historical facility data. The patient medical data is discussed further below with regard to FIGS. 9-10.

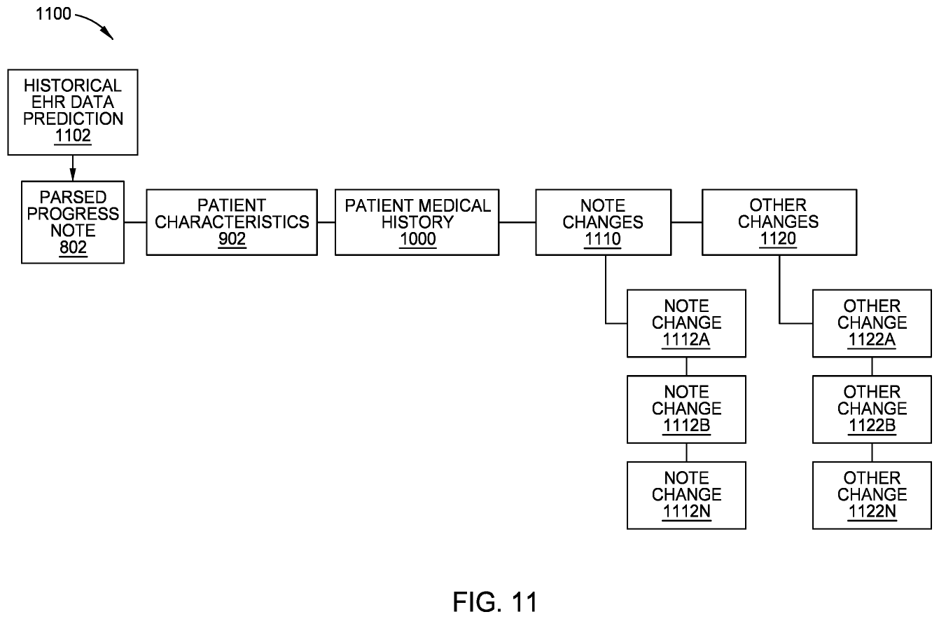

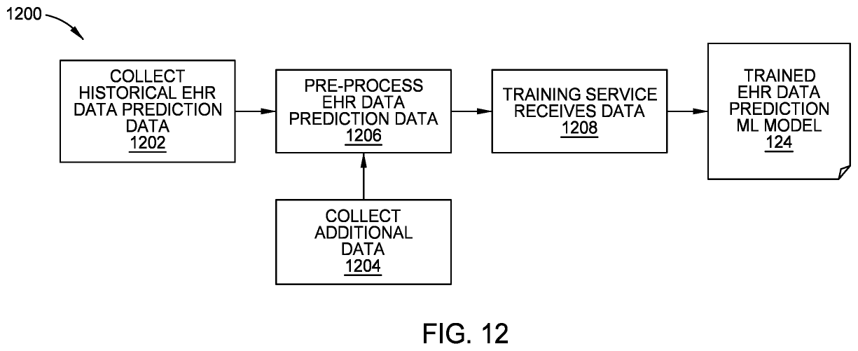

In an embodiment, the prediction service can further receive the historical progress notes data 140 illustrated in FIG. 1. This can include historical matched progress notes (e.g., the matched progress notes 142 illustrated in FIG. 1) for a variety of patients and a variety of caregivers. This is discussed further below with regard to FIG. 11. In an embodiment, the prediction service uses the historical progress notes for ongoing training of a prediction ML model (e.g., the EHR data prediction ML model 124 illustrated in FIGS. 1-2). Alternatively, the prediction service does not receive the historical progress notes data. In this example, the historical progress notes data is used to train the EHR data prediction ML model (e.g., as discussed below in relation to FIG. 12) but is not used for inference (e.g., for prediction).

At block 308, the prediction service generates an EHR data prediction (e.g., the EHR data prediction 150 illustrated in FIG. 1) from the parsed text data (e.g., generated at block 304) and the related data (e.g., identified at block 306). For example, the prediction service can provide the parsed text data and the related data to the trained prediction ML model. The trained prediction ML model can use the input data to infer an EHR data prediction. This is discussed further below with regard to FIG. 7.

In an embodiment, the EHR data prediction includes changes to the textual data. This can include identifying mistakes in the textual data (e.g., errors from input by the caregiver), identifying mis-matched patients and textual data (e.g., stemming from a caregiver selecting the wrong patient), and identifying any other suitable changes. For example, a wrong patient could be identified based on multiple inconsistencies between the textual data and other patient medical data. If the number of inconsistencies exceeds a threshold value, the EHR data prediction could indicate a likely wrong patient. In an embodiment the threshold can be pre-determined, or could be identified dynamically (e.g., using the prediction service).

In an embodiment, the EHR data prediction further includes other changes (e.g., changes to other patient data). This can include identifying other aspects of the patient's electronic health record (e.g., demographic data, diagnoses, medications, other treatments and care plans, or any other suitable data), and identifying changes or additions to those portions of the patient's electronic health record. In an embodiment the EHR data prediction both identifies the text to be changed, and the corresponding proposed change(s). This is merely an example, and the EHR data prediction can identify only the text to be changed or only the proposed changes.

In an embodiment the EHR data prediction ML model uses the parsed textual data, the patient medical data, and the historical progress notes data to generate the EHR data prediction. But this is merely an example. Alternatively, or in addition, the EHR data prediction ML model can use any subset of this data (e.g., where some of this data is unavailable for a given patient). For example, the EHR data prediction ML model can use the parsed textual data and patient medical data, without historical progress notes data. In an embodiment this may result in a slight loss of accuracy in the EHR data prediction, but the EHR data prediction is still significantly improved over prior techniques (e.g., manual prediction).

In an embodiment, the prediction service can further identify a prophylactic treatment task for the patient (e.g., a treatment task intended to quickly prevent further disease or issues for the patient). For example, the prediction service can use the parsed textual data, the patient medical data, including but not limited to specific health related data associated with one or more patients, such as age, weight, medical conditions, demographics, or other such data, or both to identify a high priority treatment task needed based on the textual data. This could be done, for example, using an additional ML model trained to use the same (or similar) inputs to the EHR data prediction model to generate a different output: a high priority treatment task, instead of an EHR data prediction. As one example, a patient progress note could identify an urgent change in medication not otherwise reflected in the patient's electronic health record. The prediction service can transmit an alert (e.g., an e-mail, SMS message, telephone call, or another form of electronic message) describing the change in medication to a care provider for the patient (e.g., to another care provider other than the care provider entering the progress notes) or to the patient themselves. The care provider or patient can then immediately implement the urgent change in medication.

As another example, a patient progress note could identify symptoms of a medical condition potentially requiring immediate medical attention (e.g., a cardiac issue). If the caregiver entering the progress note fails to provide an immediate treatment or examination, the prediction service can transmit an alert describing the potential illness to a care provider for the patient or to the patient themselves. The care provider or patient can then immediately take action to respond to the medical condition (e.g., examining the patient or seeking urgent medical care). In an embodiment, the prediction service can identify this treatment task prior to completing the EHR data prediction. For example, the prediction service can identify a high priority treatment task while generating the EHR data prediction, and can transmit the alert prior to completing EHR data prediction. In an embodiment this allows for a rapid alert for the treatment task, without waiting for complete the EHR data prediction.

At block 310, the prediction service transmits the EHR data prediction to electronic patient systems. For example, the prediction service can transmit predicted changes to the progress notes to an electronic repository (e.g., an electronic database) that maintains the progress notes. The predicted changes to the progress notes can be used to change the progress notes. As another example, the prediction service can transmit predicted other changes to an electronic system that maintains the patient's electronic health record (e.g., a care provider system). The predicted other changes can be used to change the patient's electronic health record. This is discussed further, below, with regard to FIG. 13.

### Example of Parsing Progress Notes

FIG. 4 illustrates parsing patient progress notes using ML, according to one embodiment. In an embodiment, FIG. 4 provides one example of parsing textual data using NLP, discussed above in relation to block 304 illustrated in FIG. 3. Progress notes 102 (e.g., as discussed above in relation to FIG. 1) are provided to a note parsing service 112 and a note parsing ML model 114. In an embodiment, the progress notes 102 are textual progress notes describing an examination of a patient (e.g., entered by a caregiver into an electronic system). As discussed above in relation to FIG. 1, in an embodiment, the patient progress notes 102 include a portion of unstructured textual data. For example, a caregiver may enter data in a free form textual field, in addition to (or instead of) entering data through a structured user interface (e.g., check boxes, drop-downs, and other user interface elements). This is merely an example, and the patient progress notes 102 can reflect completely unstructured textual data, partially structured textual data (e.g., data received partially from structured UI elements), or fully structured textual data (e.g., data received entirely from structured UI data).

In an embodiment, the note parsing service 112 uses the note parsing ML model 114 to parse the progress notes 102 and generate one or more parsed progress notes 410. For example, the note parsing ML model can use NLP techniques, described further below, to parse text in the progress notes 102 (e.g., unstructured text) and generate one or more parsed progress note data elements (e.g., a parsed progress note 802 as illustrated in FIG. 8, below). For example, as illustrated in FIG. 8, the parsed progress note can include patient attributes (e.g., age, height, weight, or any other suitable patient attributes), symptom attributes (e.g., one or more symptoms), diagnosis attributes (e.g., a diagnosis, onset data, resolution data, treatment, and any other suitable treatment attributes), treatment attributes (e.g., one or more medications, interventions, observations, or any other suitable treatment attributes), or any other suitable treatment attributes.

Examples may be instructive. A progress note could include the text: “RT reminded pt to watch for signs of chest tightness, chest pain, increased work of breathing & increased heart rate.” This could have been entered by a caregiver to express that a respiratory therapist (RT) reminded a patient (PT) to watch for signs of chest tightness, chest pain, increased work of breathing, and increased heartrate. The note parsing service 112 could use the note parsing ML model 114 to parse this text, and generate a parsed progress note with symptom attributes reflecting observation for chest tightness, chest pain, increased work of breathing, and increased heartrate. The parsed progress note could be, for example, a parsed progress note data element (e.g., the parsed progress note 802 illustrated below with regard to FIG. 8), with multiple symptom attributes (e.g., the symptom attributes 822A-N illustrated below with regard to FIG. 8) reflecting each of chest tightness, chest pain, increased work of breathing, and increased heartrate.

As another example, a progress note could include the text “No cough noted and pt reports sometimes coughs at night time, non-productive. Continues to keep head elevated at all times to aid in breathing.” The note parsing service 112 could use the note parsing ML model 114 to parse this text, and generate a parsed progress note with symptom attributes reflecting that the patient sometimes coughs at night, non-productive, and with treatment attributes reflecting that the patient continues to keep their head elevated at all times to aid in breathing. The parsed progress note could be, for example, a parsed progress note data element (e.g., the parsed progress note 802 illustrated below with regard to FIG. 8), with symptom attributes (e.g., the symptom attributes 822A-N illustrated below with regard to FIG. 8) reflecting a non-productive cough at night, and treatment attributes (e.g., the treatment attributes 840 illustrated below with regard to FIG. 8) reflecting that the patient continues to keep their head elevated at all times to aid in breathing.

As a final example, a progress note could include the text “Pt has made an overall improvement with respiratory status since admission, speaks in full complete sentences without noticeable dyspnea.” The note parsing service 112 could use the note parsing ML model 114 to parse this text, and generate a parsed progress note with a diagnosis attribute reflecting that the patient has made an overall improvement with respiratory status since admission. The parsed progress note could be, for example, a parsed progress note data element (e.g., the parsed progress note 802 illustrated below with regard to FIG. 8), with one or more diagnosis attributes (e.g., the diagnosis attributes 830 illustrated below with regard to FIG. 8) reflecting that the patient has made an overall improvement with respiratory status since admission. In an embodiment, these are merely examples, and the note parsing service can parse any suitable patient text.

In an embodiment, the note parsing ML model 114 can be any suitable ML model used for NLP. In an embodiment, the note parsing ML model 114 can identify any, or all, of semantic, syntax, and context information, can implement tokenization, part of speech tagging, named entity recognition, and any other suitable NLP technique, and can be used to categorize and classify the input text (e.g., the progress notes 102) to generate the parsed progress notes 410.

Further, in an embodiment the note parsing ML model 114 can be combined with one or more static rules or conditions (e.g., NLP rules or conditions that do not use an ML mode), or additional unsupervised or supervised ML models, to parse the input text. For example, the note parsing ML model 114 can be a suitable supervised ML model, and can work in concert with additional static NLP rules and conditions and one or more unsupervised ML models (e.g., using latent semantic analysis or any other suitable technique). As another example, the note parsing ML model 114 could use an annotated dictionary identifying abbreviations and common terms used in patient text (e.g., progress notes). This dictionary, to be used in concert with the ML model, could be pre-generated, generated during a training phase for the note parsing ML model 114, dynamically generated during inference of the note parsing ML model 114, or any combination thereof.

In an embodiment, the progress notes 102 can be pre-processed before being parsed using the note parsing ML model 114, or as part of being processed by the note parsing ML model. For example, the text can normalized, stemming can be used to reduce words to stem form and lemmatization can be used to derive the root form of words. These are merely examples, and any suitable pre-processing can be used.

FIG. 5 depicts an example of a table 500 of patient progress notes, according to one embodiment. In an embodiment, the table 500 is a table in an electronic database for an EHR system (e.g., a relational database, a graph database, or any other suitable electronic database). The table 500 includes numerous rows, each of which includes a row ID (e.g., identifying the row). In an embodiment, each row corresponds with a patient, designated by a patient ID, and a progress note, designated by a progress note ID.

Each row further includes a time at which the progress note was recorded (e.g., describing the time in hours, minutes, and seconds, and describing the day in month, day, and year), and a text of a progress note. For example, after a progress note is entered by a caregiver, it can be recorded in a row in the table 500. In an embodiment, the progress note text is parsed (e.g., as described above in relation to FIG. 4) after it is recorded in the table 500. Alternatively, or in addition, the progress note text is parsed after entry by a caregiver and before being recorded in the table 500.

### Example of Training a Note Parsing ML Model

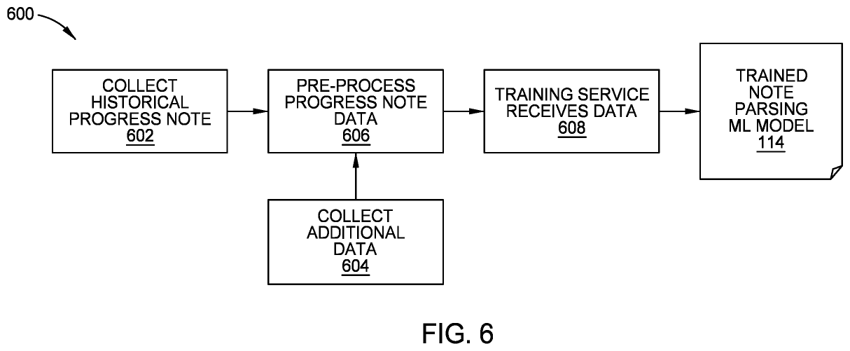

FIG. 6 is a flowchart 600 illustrating training an NLP ML model for parsing patient progress notes, according to one embodiment.

At block 602, a training service (e.g., a human administrator or a software or hardware service) collects historical progress note data. For example, a note parsing service (e.g., the note parsing service 112 illustrated in FIGS. 1-2) can be configured to act as the training service and collect historical progress note data. The historical progress note data can include, for example, a matched combination of progress note text and corresponding parsed progress note data elements generated from the text (e.g., matched progress notes 142 illustrated in FIG. 1). This is merely an example, and any suitable software or hardware service can be used (e.g., a note parsing training service).

At block 606, the training service (or other suitable service) pre-processes the collected historical progress note data. For example, the training service can create feature vectors reflecting the values of various features, for each collected matched progress note. These features can include the parsed progress note attributes illustrated in relation to FIG. 8, below, or any other suitable features. At block 608, the training service receives the feature vectors and uses them to train the note parsing ML model 114.

In an embodiment, at block 604 the training service also collects additional data (e.g., data reflecting dictionary entries for the progress note data or additional analysis of the progress note data). At block 606, the training service can also pre-process this additional data. For example, the feature vectors corresponding to the historical progress note data can be further annotated using the additional data. Alternatively, or in addition, additional feature vectors corresponding to the additional data can be created. At block 608, the training service uses the pre-processed additional data during training to generate the trained note parsing ML model 114.

In an embodiment, while a variety of suitable data is available for the training service (e.g., the historical progress note data discussed with regard to block 602 and the additional data discussed with regard to block 604), a subset of this data is selected to use for training of the note parsing ML model. That is, as part of model design the training data is selected from an available universe of training data. Further, the note parsing ML model can then use the same, or similar, data types or fields for inference for inference (e.g., as discussed above with regard to FIG. 4). This is also true of training the EHR data prediction ML model 124, as discussed below with regard to FIG. 12. Each of the supervised ML models can be trained using a selected subset of data types and fields, and the same or similar data types and fields can be used for inference.

In an embodiment, the pre-processing and training can be done as batch training. In this embodiment, all data is pre-processed at once (e.g., all historical progress note data and additional data), and provided to the training service at block 608. Alternatively, the pre-processing and training can be done in a streaming manner. In this embodiment, the data is streaming, and is continuously pre-processed and provided to the training service. For example, it can be desirable to take a streaming approach for scalability. The set of training data may be very large, so it may be desirable to pre-process the data, and provide it to the training service, in a streaming manner (e.g., to avoid computation and storage limitations). Further, in an embodiment, a federated learning approach could be used in which multiple healthcare entities contribute to training a shared model.

### Example of Predicting Progress Note Data

FIG. 7 depicts predicting electronic health record changes from parsed patient progress notes using an ML model, according to one embodiment.

In an embodiment, FIG. 7 corresponds with block 308 illustrated in FIG. 3, above. An EHR data prediction service 122, as discussed above in relation to FIGS. 1-2, is associated with an EHR data prediction ML model 124. In an embodiment, the EHR data prediction service 122 uses the EHR data prediction ML model 124 to generate one or more predicted progress note changes 720, one or more predicted changes to other systems 730, or both. In an embodiment, the EHR data prediction ML model 124 can be any suitable supervised ML model. Further, in an embodiment the EHR data prediction ML model can be made up of multiple ML models. For example, different ML models could be used to generate the predicted progress note changes and the predicted changes to other systems.

In an embodiment, the EHR data prediction service 122 uses multiple types of data to generate the predicted progress note changes 720 and predicted changes to other systems 730, using the EHR data prediction ML model 124. For example, the EHR data prediction service 122 can use one or more parsed progress note attributes 702. In an embodiment, as illustrated above in relation to FIG. 4, the parsed progress note attributes 702 are generated by a note parsing service (e.g., the note parsing service 112 illustrated in FIGS. 1-2) using a note parsing ML model (e.g., the note parsing ML model 114 illustrated in FIGS. 1-2) by parsing text data (e.g., textual progress notes).

In addition, the EHR data prediction service 122 can use patient characteristics 132 (e.g., as discussed above in relation to FIG. 1) to generate the predicted progress note changes 720 and predicted changes to other systems 730, using the EHR data prediction ML model 124. As discussed below in relation to FIG. 9, the patient characteristics 132 can include patient demographics (e.g., age, height, weight), patient medications (e.g., a listing of medications for the patient), patient assessment data (e.g., intake assessment data, discharge assessment data, activities of daily living (ADL) assessment data), or any other suitable patient characteristics.

Further, the EHR data prediction service 122 can use a patient medical history 134 (e.g., as discussed above in relation to FIG. 1) to generate the predicted progress note changes 720 and predicted changes to other systems 730, using the EHR data prediction ML model 124. As discussed below in relation to FIG. 10, the patient medical history 134 can include medical condition data (e.g., diagnosis, onset, treatment, and resolution) for any prior medical conditions.

The EHR data prediction service 122 can further use historical progress note data 140 (e.g., as discussed above in relation to FIG. 1) to generate the predicted progress note changes 720 and predicted changes to other systems 730, using the EHR data prediction ML model 124. As discussed below in relation to FIG. 11, the historical progress note data can include parsed progress note attributes (e.g., the parsed progress note 802 illustrated in FIG. 8), patient characteristics for the patient (e.g., the patient characteristics 902 illustrated in FIG. 9), patient medical data (e.g., the patient medical history 1000 illustrated in FIG. 10), note changes made to the progress note, other changes made to the patient's electronic health record, and any other suitable historical progress note data. As discussed above in relation to FIG. 1, in an embodiment the patient characteristics 132 and patient medical history 134 provide data about the particular patient for whom text is being parsed, while the historical progress note data about historical progress note and electronic health record changes for a variety of patients.

In an embodiment, the EHR data prediction service 122 uses the historical EHR data prediction data for ongoing training of the EHR data prediction ML model 124. For example, because training the EHR data prediction ML model 124 may be computationally expensive, the EHR data prediction service can train the EHR data prediction ML model 124 at suitable intervals (e.g., hourly, daily, weekly) or based on triggering events (e.g., after a threshold number of new observations are received, upon request from an administrator, or at any other suitable interval). Alternatively, the EHR data prediction service 122 does not receive the historical progress notes data 140. In this example, the historical progress notes data 140 is used to train the EHR data prediction ML model 124 (e.g., as discussed below in relation to FIG. 12) but is not used for inference (e.g., not used for generating the predicted progress note changes 720 and predicted changes to other systems 730).

In an embodiment, the predicted progress note changes 720 include a description of predicted changes to a progress note (e.g., the progress note used to generate the parsed progress note attributes 702). For example, the predicted progress note changes 720 can identify possible errors made by the caregiver entering the progress note (e.g., incorrect medications or other treatments, incorrect symptom descriptions, incorrect observational descriptions, incorrect diagnoses, or any other suitable possible error). As one example, a caregiver may include in the progress note text describing the patient's ongoing tolerance of a medication, but may incorrectly identify the medication. The predicted progress note changes 720 can identify the possible error and the likely correct medication (e.g., using the patient characteristics 132 and patient medical history 134 provided as input to the EHR data prediction ML model). As another example, a caregiver may enter progress notes for the wrong patient (e.g., using the patient characteristics 132 and patient medical history 134). The predicted progress note changes 720 can identify the potential patient mismatch, so that the progress note can be moved to the correct patient. In an embodiment, the predicted progress note changes 720 are provided automatically to an electronic patient care system, and are used to automatically change the progress notes.

In an embodiment, the predicted changes to other systems 730 include description of predicted changes to other aspects of the patient's electronic health record. For example, the parsed progress note attributes 702 may reflect a new, or different, symptom, than has been previously identified for the patient. The predicted changes to other systems 730 could identify this symptom as new, and could suggest adding the symptom to another part of the patient's electronic health record (e.g., the patient medical history 134). As another example, the parsed progress note attributes may reflect a new diagnosis, or a resolution of a previous diagnosis. The predicted changes to other systems 730 could identify this new, or resolved, diagnosis, and could suggest changes to the patient's electronic health record to reflect the change. In an embodiment, the predicted changes to other systems are provided automatically to an electronic patient care system, and are used to automatically change the patient's electronic health record.

### Example Progress Note Attributes and Patient Characteristics

FIG. 8 depicts parsed progress note data 800 for predicting electronic health record changes using an ML model, according to one embodiment. In an embodiment, the progress note data 800 provide examples for the parsed progress note attribute, illustrated in FIG. 7 and generated using a suitable note parsing ML model (e.g., the note parsing ML model 114 illustrated in FIGS. 1-2) to parse patient text (e.g., progress notes 102 illustrated in FIGS. 1 and 4). For example, the parsed progress note data 800 can include one or more parsed progress notes 802.

In an embodiment, each parsed progress notes 802 can include patient attributes 810. The patient attributes 810 can include demographic information for the patient, including one or more of an age 812, a height 814, and a weight 816. These are merely examples, and the parsed progress note 802 can include any suitable patient attributes, or no patient attributes at all (e.g., where a progress note does not describe patient attributes).

In an embodiment, each parsed progress notes 802 can further include symptom attributes 820. The symptom attributes 820 can include attributes of one or more symptoms 822A-N experienced by the patient and described in the progress note. These are merely examples, and the parsed progress note 802 can include any suitable symptom attributes, or no symptom attributes at all (e.g., where a progress note does not describe patient symptoms).

In an embodiment, each parsed progress notes 802 can further include one or more diagnosis attributes 830. The diagnosis attributes 830 can include a diagnosis 832 (e.g., a label or description for the diagnosis), an onset description 834 (e.g., a date of onset or textual description), a treatment 836 (e.g., a treatment history for the diagnosed medical condition), and a resolution 838 (e.g., a date of resolution or a notation that the diagnosed medical condition is ongoing). These are merely examples, and the parsed progress note 802 can include any suitable diagnosis attributes, or no diagnosis attributes at all (e.g., where a progress note does not describe a patient diagnosis).

In an embodiment, each parsed progress notes 802 can further include one or more treatment attributes 840. The treatment attributes 840 can include a medication 842 (e.g., a label or description for the medication), an intervention description 846 (e.g., a textual description of medical procedures or interventions performed as part of treatment), and one or more observations (e.g., descriptions of observations of treatment). In an embodiment, the treatment attributes 840 provide additional detail for a treatment 836 included in a diagnosis attribute 830. Alternatively, or in addition, the treatment attributes 840 are separate from the diagnosis attributes 830. These are merely examples, and the parsed progress note 802 can include any suitable treatment attributes, or no treatment attributes at all (e.g., where a progress note does not describe a patient treatment).

FIG. 9 depicts patient characteristics 900 for use in predicting electronic health record changes from parsed patient progress notes using an ML model, according to one embodiment. In an embodiment, the patient characteristics 900 provide examples for the patient characteristics 132, described above in relation to FIG. 1. A patient 902 includes patient demographics 910. For example, the patient demographics 910 can include age 912, height 914, and weight 916. These are merely examples, and the patient demographics 910 can include any suitable characteristics.

The patient 902 can further include patient medications 920. In an embodiment, the patient medications 920 include one or more medications 922A-N. These are merely examples, and the patient medications 920 can include any suitable data.

Further, the patient 902 can include one or more patient assessments 930 (e.g., a patient assessment 930 corresponding to each healthcare facility to which the patient has been admitted). In an embodiment, the patient assessment 930 includes an intake assessment 932. For example, an intake assessment can be performed for the patient upon intake to a healthcare facility (e.g., performed by a suitable healthcare professional, using a suitable automated assessment system, or both). The intake assessment can be memorialized as the intake assessment 932.

In an embodiment, the patient assessment 930 further includes a discharge assessment 934. For example, a discharge assessment can be performed for the patient upon discharge from a healthcare facility (e.g., performed by a suitable healthcare professional, using a suitable automated assessment system, or both). The discharge assessment can be memorialized as the discharge assessment 934.

The patient assessment 930 can further include an activities of daily living (ADL) assessment 936. For example, the ADL assessment can memorialize the patient's ability to dress, feed, ambulate, toilet, and perform their own hygiene. The ADL assessment can be memorialized as the ADL assessment 936. These are merely examples, and the patient assessment 930 can include any suitable data. Further, the patient demographics 910, patient medications 920, and patient assessment 930 are merely examples. The patient 902 can include any suitable patient data, organized in any suitable fashion.

FIG. 10 depicts patient medical history 1000 for use in predicting electronic health record changes from parsed patient progress notes using an ML model, according to one embodiment. In an embodiment, the patient medical history 1000 provide examples for the patient medical history 134, described above in relation to FIG. 1.

A patient 1002 includes one or more medical conditions 1010A-N. Each medical condition includes a respective diagnosis 1012A-N, a respective onset description 1014A-N (e.g., a date or textual description), a respective treatment 1016A-N (e.g., a treatment history for the medical condition), and a respective resolution 1018A-N (e.g., a date of resolution or a notation that the medical condition is ongoing). These are merely examples, and each medical condition 1010A-N can include any suitable data. Further, the medical conditions 1010A-N are merely examples, and the patient 1002 can include any suitable medical history data.

FIG. 11 depicts historical patient progress note data 1100 for use in predicting electronic health record changes from parsed patient progress notes using an ML model, according to one embodiment. In an embodiment, a historical EHR data prediction 1102 includes one or more parsed progress notes 802 (e.g., as discussed above in relation to FIG. 8), one or more patient characteristics 920 (e.g., as discussed above in relation to FIG. 9), and patient medical history 1000 (e.g., as discussed above in relation to FIG. 10).

In an embodiment, the historical EHR data prediction 1102 further includes note changes 1110. For example, the note changes 1110 can include one more note changes 1112A-N made to a progress note (e.g., the progress note corresponding to the parsed progress note 802). For example, the note changes 1112A-N can describe historical progress note changes made to the progress note based on the parsed progress note 802, patient characteristics 902, and patient medical history 1000. These are merely examples, and the historical EHR data prediction 1102 can include any suitable note changes, or no note changes at all (e.g., where no changes were made to the progress note).

In an embodiment, the historical EHR data prediction 1102 further includes other changes 1120. For example, the other changes 1120 can include one more other changes 1122A-N made to a patient's electronic health record. For example, the other changes 1122A-N can describe historical changes made to the patient's electronic health record based on the parsed progress note 802, patient characteristics 902, and patient medical history 1000. These are merely examples, and the historical EHR data prediction 1102 can include any suitable other changes, or no other changes at all (e.g., where no changes were made to the electronic health record). In an embodiment, as discussed above, an EHR data prediction ML model can include multiple ML models. For example, one ML model can be trained to generate progress note changes (e.g., reflected in the note changes 1110) and another ML model can be trained to generate other changes (e.g., reflected in the other changes 1120).

### Example of Training an ML Model for EHR Data Prediction

FIG. 12 is a flowchart 1200 illustrating training an ML model for predicting electronic health record changes from parsed patient progress notes using an ML model, according to one embodiment. At block 1202, a training service (e.g., a human administrator or a software or hardware service) collects historical EHR data prediction data (e.g., the historical EHR data prediction data 1100 illustrated in FIG. 11). For example, an EHR data prediction service (e.g., the EHR data prediction service 122 illustrated in FIGS. 1-2) can be configured to act as the training service and collect historical EHR data prediction data. This is merely an example, and any suitable software or hardware service can be used (e.g., an EHR data prediction training service).

At block 1206, the training service (or other suitable service) pre-processes the collected historical EHR data prediction data. For example, the training service can create feature vectors reflecting the values of various features, for each historical EHR data prediction. At block 1208, the training service receives the feature vectors and uses them to train the note parsing ML model 114.

In an embodiment, at block 1204 the training service also collects additional data (e.g., data reflecting additional observations or data about the patient). At block 1206, the training service can also pre-process this additional data. For example, the feature vectors corresponding to the historical EHR data prediction data can be further annotated using the additional data. Alternatively, or in addition, additional feature vectors corresponding to the additional data can be created. At block 1208, the training service uses the pre-processed additional data during training to generate the trained EHR data prediction ML model 124.

In an embodiment, as discussed above in relation to FIG. 6, the pre-processing and training can be done as batch training or in a streaming manner. Further, a federated learning approach could be used.

FIG. 13 depicts using predicted electronic health record changes, according to one embodiment. In an embodiment, a prediction controller 1310 (e.g., the prediction controller 200 illustrated in FIG. 2) generates predicted note changes 1320, predicted other changes 1322, or both. For example, as discussed above in relation to block 308 in FIG. 3 and FIG. 7, an EHR data prediction service (e.g., the EHR data prediction service 122 illustrated in FIGS. 1-2) can use an EHR data prediction ML model (e.g., EHR data prediction ML model 124 illustrated in FIGS. 1-2) to predict note changes and other changes to a patient's electronic health record.

For example, the EHR data prediction service can use parsed progress note attributes, generated using a note parsing service (e.g., the note parsing service 112 illustrated in FIGS. 1-2) and a note parsing ML model (e.g., the note parsing ML model 114 illustrated in FIGS. 1-2) from patient text data (e.g., the patient progress notes 102 or other text data 104 illustrated in FIG. 1). As discussed above, FIG. 8 provides an example of parsed progress note attributes. The EHR data prediction service can further use any, or all, of patient characteristics (e.g., as illustrated in FIG. 9), patient medical history (e.g., as illustrated in FIG. 10), and historical progress notes data (e.g., as illustrated in FIG. 11).

In an embodiment, the prediction controller 1310 transmits the predicted note changes 1320, the predicted other changes 1322, or both, over a communication network 1330 to any, or all, of a patient 1340, a care provider 1350, and a healthcare facility 1360. The communication network 1330 can be any suitable communication network.

In an embodiment, any, or all, of the care provider 1350 and the healthcare facility 1360 receive the predicted note changes 1320, the predicted other changes 1322, or both. The predicted note changes 1320, the predicted other changes 1322, or both, can then be used to treat the patient. For example, the predicted changes can be used to ensure that the patient receives the correct treatment, ensure that the patient is correctly monitored for symptoms, and otherwise treat the patient.

In an embodiment, the prediction controller 1310 can interact directly with a care provider 1350 or healthcare facility 1360 to apply the predicted note changes 1320, the predicted other changes 1322, or both. For example, the prediction controller 1310 can interact directly with electronic systems of a care provider 1350 or healthcare facility 1360 (e.g., using a suitable application programming interface (API), web interface, or other electronic interface) to implement predicted changes. This can include updating the patient's electronic health record to reflect the changes, and providing suitable alerts to the patient or caregiver reflecting the changes. In one embodiment, the care provider 1350 or healthcare facility 1360 implements some, or all, of the predicted note changes 1320 and predicted other changes 1322 automatically. Alternatively, or in addition, the care provider 1350 or healthcare facility 1360 provides the predicted changes to a suitable caregiver, for consideration and implementation.

As another example, the patient 1340 can receive predicted other changes 1322 (e.g., a change to the patient's medication or care plan) at a suitable electronic device (e.g., a smartphone, tablet, laptop computer, desktop computer, or any other suitable device). The patient 1340 can use the predicted other changes 1322 to select or improve treatment (e.g., using a mobile application or local application running on the patient device, or accessing the predicted other changes 1322 over the communication network 1330).

In an embodiment, any, or all, of patient 1340, the care provider 1350, and the healthcare facility 1360 store the predicted note changes 1320 and predicted other changes 1322. For example, this can allow the recipient to access the predicted note changes 1320 and predicted other changes 1322 without requiring a continuous network connection.

Further, in an embodiment, the prediction controller 1310 can transmit an alert to the healthcare facility 1360, care provider 1350, or patient 1340. For example, as described above in relation to FIG. 3, the prediction controller can further include an additional ML model trained use the same (or similar) inputs to the EHR data prediction model to identify a high priority treatment task (e.g., a prophylactic treatment task, including an urgent change in medication, an urgent medical condition, or any other high priority treatment task), instead of an EHR data prediction. The prediction controller can transmit the alert 1324 (e.g., an e-mail, SMS message, telephone call, or another form of electronic message) describing the high priority treatment task to any, or all, of the healthcare facility 1360, care provider 1350, or patient 1340.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
determining a plurality of attributes of a textual description of a patient medical examination, comprising:
detecting the plurality of attributes based on analyzing the textual description using a first machine learning (ML) model trained to parse patient textual descriptions; and
predicting a change to an electronic health record for the patient, comprising:
providing to a second ML model the plurality of attributes of the textual description and patient medical data for the patient, wherein the second ML model is trained to predict changes to patient electronic health records based on attributes of textual data relating to the patient and patient medical data,
wherein the predicted change is provided to an electronic system to change the electronic health record and affect medical treatment for the patient.

2. The method of claim 1, wherein the predicting the change to the electronic health record comprises predicting a change to the textual description of the patient medical examination.

3. The method of claim 2, wherein the predicting the change to the textual description comprises identifying an error for the textual description, based on the patient medical data.

4. The method of claim 3, wherein the error for the textual description comprises the textual description being associated with a wrong patient.

5. The method of claim 4, further comprising:
removing the association of the textual description with the wrong patient using the electronic system.

6. The method of claim 4, further comprising:
determining that the textual description is associated with a wrong patient based on comparing a number of inconsistencies between the textual description and electronic health record to a threshold value.

7. The method of claim 1, wherein the predicting the change to the electronic health record comprises predicting a change to at least one of a: (i) symptom, (ii) diagnosis, or (iii) treatment for the patient recorded in the electronic health record for the patient.

8. The method of claim 7, further comprising:
updating the electronic health record to reflect the change the at least one of the: (i) symptom, (ii) diagnosis, or (iii) treatment for the patient using the electronic system.

9. The method of claim 1, further comprising:
identifying a prophylactic treatment task for the patient based on the plurality of attributes of the textual description; and
transmitting an electronic alert relating to the treatment task.

10. An apparatus comprising:
a memory; and
a hardware processor communicatively coupled to the memory, the hardware processor configured to perform operations comprising:
determining a plurality of attributes of a textual description of a patient medical examination, comprising:
detecting the plurality of attributes based on analyzing the textual description using a first machine learning (ML) model trained to parse patient textual descriptions; and
predicting a change to an electronic health record for the patient, comprising:
providing to a second ML model the plurality of attributes of the textual description and patient medical data for the patient, wherein the second ML model is trained to predict changes to patient electronic health records based on attributes of textual data relating to the patient and patient medical data,
wherein the predicted change is provided to an electronic system to change the electronic health record and affect medical treatment for the patient.

11. The apparatus of claim 10, wherein the predicting the change to the electronic health record comprises predicting a change to the textual description of the patient medical examination.

12. The apparatus of claim 11, wherein the predicting the change to the textual description comprises identifying an error for the textual description, based on the patient medical data.

13. The apparatus of claim 12, wherein the error for the textual description comprises the textual description being associated with a wrong patient.

14. The apparatus of claim 10, wherein the predicting the change to the electronic health record comprises predicting a change to at least one of a: (i) symptom, (ii) diagnosis, or (iii) treatment for the patient recorded in the electronic health record for the patient.

15. The apparatus of claim 10, the operations further comprising:
identifying a prophylactic treatment task for the patient based on the plurality of attributes of the textual description; and
transmitting an electronic alert relating to the treatment task.

16. A non-transitory computer-readable medium comprising instructions that, when executed by a processor, cause the processor to perform operations comprising:
determining a plurality of attributes of a textual description of a patient medical examination, comprising:
detecting the plurality of attributes based on analyzing the textual description using a first machine learning (ML) model trained to parse patient textual descriptions; and
predicting a change to an electronic health record for the patient, comprising:
providing to a second ML model the plurality of attributes of the textual description and patient medical data for the patient, wherein the second ML model is trained to predict changes to patient electronic health records based on attributes of textual data relating to the patient and patient medical data,
wherein the predicted change is provided to an electronic system to change the electronic health record and affect medical treatment for the patient.

17. The non-transitory computer-readable medium of claim 16, wherein the predicting the change to the electronic health record comprises identifying an error for the textual description, based on the patient medical data.

18. The non-transitory computer-readable medium of claim 17, wherein the error for the textual description comprises the textual description being associated with a wrong patient.

19. The non-transitory computer-readable medium of claim 16, wherein the predicting the change to the electronic health record comprises predicting a change to at least one of a: (i) symptom, (ii) diagnosis, or (iii) treatment for the patient recorded in the electronic health record for the patient.

20. The non-transitory computer-readable medium of claim 16, the operations further comprising:
identifying a prophylactic treatment task for the patient based on the plurality of attributes of the textual description; and
transmitting an electronic alert relating to the treatment task.
